(19) United States
(12) Patent Application Publication
(10) Pub. No.: US 2025/0387530 A1
(43) Pub. Date: Dec. 25, 2025

(54) RADIOPAQUE MONOMER AND EMBOLISATION MICROSPHERES COMPRISING SAME

(71) Applicants: GUERBET, VILLEPINTE (FR); CENTRE NATIONAL DE LA RECHERCHE SCIENTIFIQUE (CNRS), PARIS (FR); UNIVERSITE PARIS-SACLAY, GIF-SUR-YVETTE (FR)

(72) Inventors: Laurence MOINE, SAINT CLOUD (FR); Baptiste François, PARIS (FR); Olivier FOUGERE, MORTEFONTAINE (FR); Stéphanie Louguet, GRADIGNAN (FR)

(73) Assignees: GUERBET, VILLEPINTE (FR); CENTRE NATIONAL DE LA RECHERCHE SCIENTIFIQUE (CNRS), PARIS (FR); UNIVERSITE PARIS-SACLAY, GIF-SUR-YVETTE (FR)

(21) Appl. No.: 18/879,667

(22) PCT Filed: Jun. 28, 2023
(86) PCT No.: PCT/EP2023/067727

§ 371 Date: Dec. 27, 2024

(30) Foreign Application Priority Data
Jun. 28, 2022 (EP) 22305941.1

(51) Int. Cl. A61L 24/00; A61K 49/04; C07C 69/54

(52) U.S. Cl. CPC A61L 24/001; A61K 49/0438; A61L 24/0068; C07C 69/54

(57) ABSTRACT

The present invention relates to a compound of the following formula (A) mainly for use as a radiopaque monomer: The invention further relates to radiopaque embolisation microspheres based at least on: 20% to 90% hydrophilic monomer; 5% to 50% compound of formula (A); 1% to 15% non-biodegradable hydrophilic crosslinking monomer; and −0.1% to 10% transfer agent. The invention also relates to a pharmaceutical composition comprising at least one embolisation microsphere according to the invention, in association with a pharmaceutically acceptable carrier, advantageously for parenteral administration. The invention further relates to a kit comprising a pharmaceutical composition according to the invention in association with a pharmaceutically acceptable carrier for parenteral administration, and to an injection means.

### FIELD OF THE INVENTION

The present invention relates to a novel halogenated radiopaque monomer, intended in particular to be used within a crosslinked matrix participating in the composition of embolization microspheres.

### PRIOR ART

Therapeutic vascular occlusion (that is to say, embolization) is used to prevent or to treat certain pathological conditions in situ. It can be carried out by means of catheters making it possible, under imaging control, to position particulate occlusion agents (that is to say, emboli or embolic agents) in the circulatory system. It has a variety of medical applications, such as the treatment of vascular malformations, hemorrhagic processes, or tumors, including, for example, uterine fibromas, primary or secondary liver tumors. For example, vascular occlusion may cause a tumoral necrosis and avoid a more invasive operation. This occlusion technique may also be coupled to delivery of an anticancer agent in the context of chemoembolization. This makes it possible to increase the local concentration while limiting the systemic exposure of a medicinal product by a targeted injection, as well as its residence time in the tumor. In the case of vascular malformations, vascular occlusion makes it possible to normalize the blood flow to normal tissues, and to aid surgery while limiting the risk of hemorrhage. In hemorrhagic processes, vascular occlusion may lead to a decrease in flow rate, which promotes healing of the arterial wound. Furthermore, depending on the pathologies treated, embolization may be used for temporary purposes or for permanent purposes.

Embolization agents are conventionally introduced into a blood vessel via a catheter, in particular a microcatheter, the diameter of which is less than that of the vessel to be treated. Embolization agents for vascular occlusion comprise, for example, embolization liquids (acrylic adhesives, gels), mechanical devices, polymeric embolization microspheres and particles. The choice of a specific material depends on many factors, such as the type of lesion to be treated, the type of catheter to be used and the need for temporary or permanent embolization.

Embolization microspheres based on polymers are particularly useful for the abovementioned therapeutic purposes. They can be biodegradable for temporary embolization, as described in the applications WO 2012/120139 and WO 2012/120138, or nonbiodegradable for permanent embolization.

For example, the Embosphere® product (Biosphere Medical) corresponds to nonbiodegradable microspheres based on trisacryl (N-acryloyl-2-amino-2-hydroxymethylpropane-1,3-diol) and on gelatin. Nonbiodegradable microspheres based on acrylic copolymers and on polyvinyl alcohol (PVA) have also been proposed for permanent embolization (Osuga et al. (2002), J. Vasc. Interv. Radiol., 13, 929-34).

In addition, in order to be visible in X-ray imaging, embolization microspheres can be rendered radiopaque by addition of a radiopaque monomer or entity to their composition. Such radiopaque embolization microspheres are described in the applications WO 2021/069527 and WO 2021/069528. The microspheres of these applications incorporate a radiopaque monomer denoted MAOETIB, of following formula:

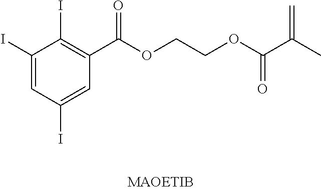 [SMILES: C=C(C)C(=O)OCCOC(=O)c1cc(I)cc(I)c1I]

Radiopacity refers to the relative inability of electromagnetism, in particular of X-rays, to pass through dense materials, which are described as “radiopaque”, appearing opaque/white in a radiography image. Bearing in mind the complexity of the content in a radiographic or fluoroscopic image, clinicians are sensitive to the quality of the image as regards the luminosity or the power of the signal from the material in the image. The two main factors which contribute to the level of the radiopacity are the density and the atomic number. Medical devices based on polymers requiring radiopacity typically use a mixture of polymers which incorporates a small amount, as percentage by weight, of a radiopaque element, such as, for example, a heavy atom, such as a halogen, in particular iodine. The ability of a device to be visualized by fluoroscopy depends on the amount or on the density of the radiopaque element mixed in the material. However, the addition of a radiopaque monomer or entity having halogenated groups appears to considerably reduce the hydrophilic nature of the material. In addition, the microspheres incorporating this type of radiopaque monomer or entity experience an increase in their density, which impacts their properties of suspendability in the injection medium. To sum up, the microspheres charged with iodine in order to be visible under X-rays of the prior art are typically more hydrophobic, dense and rigid than the microspheres not visible under X-rays and have a tendency to form microsphere aggregates. Consequently, (1) they are difficult to keep in suspension during the duration of the injection into the catheter, (2) they often block the catheter, even when their diameter is less than the internal diameter of the catheter (Duran 2016), for example owing to the fact that they agglomerate more easily together, and (3) they have a tendency to stick to the walls of the catheter.

It is thus preferable to have available radiopaque monomers or entities participating in the composition of embolization microspheres which make it possible for the latter to remain hydrophilic and flexible when they are swollen with water. It is also desired for these microspheres to exhibit mechanical properties, in particular a degree of swelling, an elasticity and a compressibility, which are appropriate for injection via a catheter or a microcatheter. It is also desired for these microspheres to be able to be kept in suspension in the injection mixture (mixture composed of contrast product and of aqueous phase) throughout the duration of the injection into the catheter. This is because, in order to be injectable and in order for the practitioner to be able to monitor the injection under X-ray control, the microspheres are generally suspended in a mixture of nonionic iodinated contrast product and of aqueous phase. For this, radiologists generally use a solution of contrast product and optionally of physiological saline, of bicarbonate buffer of or phosphate buffer, advantageously a solution of 100% of contrast product. To guarantee their injectability, the microspheres have to be kept in suspension homogeneously in this solution. If the microspheres settle out or, on the contrary, float at the surface of the solution, the resulting suspension is nonhomogeneous and unstable and thus cannot be injected into the patient.

The applications WO 2021/069527 and WO 2021/069528 describe halogenated radiopaque monomers which make it possible to satisfactorily meet these requirements. However, the need remains for novel radiopaque monomers or entities intended for the preparation of embolization microspheres which make it possible to obtain better performance qualities, for example in terms of stability or of injectability of suspensions comprising said microspheres, while remaining compatible with the iodinated contrast products as described above.

### SUMMARY OF THE INVENTION

In this context, the inventors have developed a novel halogenated radiopaque monomer of formula (A) resulting in an improvement in the performance qualities of embolization microspheres comprising this monomer in their composition. For example, this novel radiopaque monomer of formula (A) makes it possible in particular for the embolization microspheres comprising it to avoid aggregating together in the catheter or the microcatheter before injection. The presence of this novel halogenated radiopaque monomer of formula (A) in embolization microspheres also prevents the latter from sticking to the walls of the catheter or of the microcatheter before injection. In addition, the properties of suspendability and of injectability of these microspheres are improved by virtue of this novel radiopaque monomer.

The expression “improved suspendability” is understood to mean, within the meaning of the present invention, the ability of the microspheres to form a suspension which is stable over a time compatible with their use and which is homogeneous, that is to say with an identical distribution of the microspheres at any point in the volume of the suspension.

The expression “improved properties of injectability” is understood to mean, within the meaning of the present invention, the ability of the suspension to be injected via an injection system, such as a syringe or a catheter, without creating blockages and without requiring significant force on the part of the practitioner.

The present invention thus relates to a compound of following formula (A):

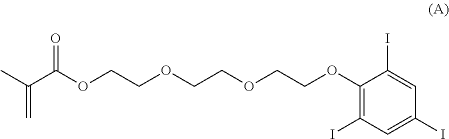 [SMILES: C=C(C)C(=O)OCCOCCOCCOc1c(I)cc(I)cc1I]

This compound is also denoted by the term MAETIP in the present description.

Another subject matter of the present invention relates to the use of the compound of formula (A) as defined above as halogenated radiopaque monomer.

Another subject matter of the invention relates to embolization microspheres comprising said halogenated radiopaque monomer of formula (A).

The present invention thus also relates to the use of this compound of formula (A) in embolization microspheres.

The present invention additionally relates to a pharmaceutical composition comprising embolization microspheres as defined above, in combination with a pharmaceutically acceptable vehicle, advantageously for administration by injection.

Another subject matter of the present invention is a kit comprising a pharmaceutical composition as defined above and at least one means of injection of said composition, for administration of said composition parenterally.

Another subject matter of the present invention is a kit comprising, on the one hand, a pharmaceutical composition as defined above and, on the other hand, a contrast agent for imaging by X-ray, by magnetic resonance or by ultrasonography, and optionally at least one means of injection for administration parenterally; advantageously, said means of injection is the Vectorio® device as described in the applications WO2016/166346, WO2016/166339, WO2017/005914 and WO2017/081178.

### DETAILED DESCRIPTION

The main subject matter of the present invention is thus the compound of following formula (A):

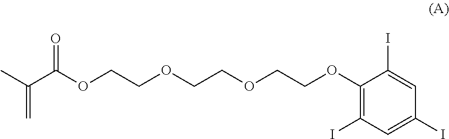 [SMILES: C=C(C)C(=O)OCCOCCOCCOc1c(I)cc(I)cc1I]

In the compound of formula (A), the iodine atoms are placed in the 2, 4 and 6 positions of the phenyl ring. Due to the size of the iodine atoms and to their homogeneous distribution on the phenyl ring, this compound exhibits a reduced spatial accessibility to the aromatic carbons (in the 3 and 5 positions of the phenyl ring), in comparison with MAOETIB or with the compound (Vb) of the application WO 2021/069528 (where the iodine atoms are in the 2, 3 and 5 positions and the aromatic carbons are in the 4 and 6 positions of the phenyl ring). The restricted accessibility to the aromatic carbons in the compound of formula (A) appears to have the effect of reducing the lipophilic nature of the molecule. This is because the inter- or intramolecular interactions of these carbons are reduced, indeed even eliminated, so as to limit the sticky nature of the molecule. In other words, the spatial configuration of the compound of formula (A) makes it possible to limit the aggregation together of the embolization microspheres incorporating said compound.

According to the present invention, this compound of formula (A) is advantageously used as halogenated radiopaque monomer. Thus, another subject matter of the present invention is the use of the compound of formula (A) as defined above as halogenated radiopaque monomer.

In addition, the present invention relates to embolization microspheres comprising said halogenated radiopaque monomer of formula (A). In particular, said embolization microspheres comprise a crosslinked polymeric matrix comprising the halogenated radiopaque monomer of formula (A).

In a particular embodiment, said crosslinked polymeric matrix is as defined in the application WO2021/069528, with the exception of the halogenated radiopaque monomer of general formula (II) replaced by the compound of formula (A) according to the invention. In other words, said crosslinked polymeric matrix is based on at least:
a) from 20% to 90% of hydrophilic monomer chosen from N-vinylpyrrolidone and a monomer of following formula (I):

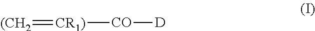

in which:
   D represents O—Z or NH—Z, Z representing (C₁-C₆)alkyl, —(CR₂R₃)ₘ—CH₃, —(CH₂—CH₂—O)ₘ—H, —(CH₂—CH₂—O)ₘ—CH₃, —C(R₄OH)ₘ or —(CH₂)ₘ—NR₅R₆ with m representing an integer from 1 to 30; preferably, m is equal to 4 or 5;
   R₁, R₂, R₃, R₄, R₅ and Re represent, independently of one another, H or a (C₁-C₆)alkyl;
b) from 5% to 50% of compound of following formula (A):

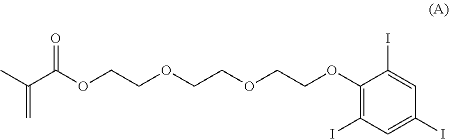 [SMILES: C=C(C)C(=O)OCCOCCOCCOc1c(I)cc(I)cc1I]

c) from 1% to 15% of nonbiodegradable linear or branched hydrophilic crosslinking monomer exhibiting (CH₂═(CR₁₆))— groups at each of its ends, each R₁₆ independently representing H or a (C₁-C₆)alkyl; and
d) from 0.1% to 10% of transfer agent chosen from alkyl halides and cycloaliphatic or aliphatic thiols in particular having from 2 to 24 carbon atoms, and optionally having another functional group chosen from the amino, hydroxy and carboxy groups,
the percentages of the monomers a) to c) being given in moles, with respect to the total number of moles of monomers, and the percentages of the compound d) being given in moles, with respect to the number of moles of the hydrophilic monomer a).

The hydrophilic monomer of formula (I), the crosslinking monomer c) and the transfer agent d) are advantageously as defined in the application WO2021/069528, in particular on pages 13 and 19-22.

The term “hydrophilic monomer” is understood to mean, within the meaning of the present invention, a monomer having a strong affinity for water, that is to say tending to dissolve in water, to mix with water, to be wetted by water, or capable of swelling in water after polymerization.

The term “crosslinking monomer” is understood to mean, within the meaning of the present invention, an at least bifunctional but also multifunctional monomer possessing a double bond at each polymerizable end. The crosslinking monomer, in combination with the other monomers in the mixture, makes possible the formation of a crosslinked network. The structure and the amount of crosslinking monomer(s) in the mixture of monomers can be easily chosen by a person skilled in the art in order to provide the desired crosslinking density. The crosslinking agent is also advantageous for the stability of the microspheres. The crosslinking agent prevents the microspheres from being able to dissolve in any solvent. The crosslinking agent also makes it possible to improve the compressibility of the microspheres, which is favorable to embolization.

The term “nonbiodegradable hydrophilic crosslinking agent” is understood to mean, within the meaning of the present invention, a crosslinking agent as defined above, having a strong affinity for water and not being able to be degraded under the physiological conditions of the body of a mammal, in particular the human body. This is because the biodegradation of a molecule is made possible when the latter contains sufficient functional sites which can be cleaved under physiological conditions, in particular by the endogenous enzymes of the body of a mammal, in particular of the human body, and/or at physiological pH (generally in the vicinity of 7.4). The functional sites which can be cleaved under physiological conditions are in particular amide bonds, ester bonds and acetals. A molecule comprising an insufficient number of said functional sites will thus be regarded as nonbiodegradable. In the context of the present invention, the crosslinking monomer contains less than 20 functional sites which can be cleaved under physiological conditions, preferably less than 15 sites, more preferably less than 10 sites, more preferably still less than 5 sites.

In the context of the present invention, the term “transfer agent” is understood to mean a chemical compound possessing at least one weak chemical bond. This agent reacts with the radical site of a growing polymer chain and interrupts the growth of the chain. In the chain transfer process, the radical is transferred temporarily to the transfer agent, which restarts the growth by transferring the radical to another polymer or monomer.

The expression “matrix based on” should of course be understood as meaning a matrix comprising the mixture of and/or the product of the reaction between the base constituents used for the polymerization in a heterogeneous medium of this matrix, preferably only the product of the reaction between the different base constituents used for this matrix, it being possible for some of them to be intended to react or to be capable of reacting together or with their close chemical environment, at least partly, during the different phases of the process for the manufacture of the matrix, in particular during a polymerization stage. Thus, the base constituents are the reactants intended to react together during the polymerization of the matrix. The base constituents are therefore introduced into a reaction mixture optionally additionally comprising a solvent or a mixture of solvents and/or other additives, such as at least one salt and/or at least one polymerization initiator and/or at least one stabilizer, such as PVA. In the context of the present invention, the reaction mixture comprises at least the monomers a), b), c) and the transfer agent d) mentioned in the present description as base constituents, optionally a polymerization initiator, such as, for example, t-butyl peroxide, benzoyl peroxide, azobiscyanovaleric acid (also called 4,4′-azobis(4-cyanopentanoic acid)), AIBN (azobisisobutyronitrile), or 1,1′-azobis(cyclohexanecarbonitrile) or one or more thermal initiators, such as 2-hydroxy-4′-(2-hydroxyethoxy)-2-methylpropiophenone (106797-53-9); 2-hydroxy-2-methylpropiophenone (Darocur® 1173, 7473-98-5); 2,2-dimethoxy-2-phenylacetophenone (24650-42-8); 2,2-dimethoxy-2-phenylacetophenone (Irgacure®, 24650-42-8) or 2-methyl-4′-(methylthio)-2-morpholinopropiophenone (Irgacure®, 71868-10-5), and at least one solvent, preferably a solvent mixture comprising an aqueous solvent and an organic solvent, such as a nonpolar aprotic solvent, for example an immiscible water/toluene system.

Thus, according to the present invention, the matrix is at least based on the monomers a), b), c) and on the transfer agent d) mentioned in the present description, these compounds therefore being base constituents.

Thus, in the present description, the expressions similar to “the [base constituent X] is in particular added to the reaction mixture in an amount of from YY % to YYY %” and to “the crosslinked matrix is in particular based on the [base constituent X] in an amount of from YY % to YYY %” interpreted are similarly. Likewise, the expressions similar to “the reaction mixture comprises at least [the base constituent X]” and to “the crosslinked matrix is based on at least [the base constituent X]” are interpreted similarly.

The term “organic phase” of the reaction mixture is understood to mean, within the meaning of the present invention, the phase comprising the organic solvent and the compounds in said organic solvent, in particular the monomers, the transfer agent and the polymerization initiator.

The term “(C_{X}-C_{Y})alkyl” group is understood to mean, within the meaning of the present invention, a saturated, or monovalent linear branched, hydrocarbon chain comprising from X to Y carbon atoms, X and Y being integers of between 1 and 36, preferably 1 and 18, in particular 1 and 6. Mention may be made, by way of example, of the methyl, ethyl, propyl, isopropyl, butyl, isobutyl, sec-butyl, tert-butyl, pentyl or hexyl groups.

In the context of the present invention, the compound of formula (A) is in particular added to the reaction mixture in an amount of from 5% to 50%, in particular in an amount of greater than 7% and of less than or equal to 50%, in particular in an amount of greater than 10% and of less than or equal to 50%, more particularly in an amount of greater than 15% and of less than or equal to 50%, preferably in an amount of greater than 15% and of less than or equal to 35%, and in particular of from 20% to 30%, per mole, with respect to the total number of moles of monomers.

The embolization microspheres comprising the crosslinked polymeric matrix as defined above advantageously correspond to spherical particles having a diameter after swelling ranging from 20 to 1200 μm, for example from 20 to 100 μm, from 40 to 150 μm, from 100 to 300 μm, from 300 to 500 μm, from 500 to 700 μm, from 700 to 900 μm or from 900 to 1200 μm, as determined by optical microscopy. The microspheres advantageously have a small enough diameter to be injected by needles, a catheter or a microcatheter with an inside diameter varying from a few hundred micrometers to more than one millimeter.

The expression “after swelling” means that the size of the microspheres is considered after the polymerization and sterilization stages which take place during their preparation. The sterilization stage involves, for example, passage of the microspheres, after the polymerization stage, through an autoclave at high temperature, typically at a temperature of greater than 100° C., preferably at a temperature of between 110° C. and 150° C., preferably 121° C. During this sterilization stage, the microspheres continue to swell in a controlled way, that is to say with a managed degree of swelling. The degree of swelling is defined as:

$$degree of swelling by weight ( Q ) = \frac{{w}_{w} ( g ) - {w}_{d} ( g )}{{w}_{d} ( g )}$$

where w_{w} is the weight in grams of 1 ml of sedimented microspheres and w_{d} is the weight in grams of 1 ml of sedimented microspheres which have subsequently been lyophilized.

In a particular embodiment according to the invention, the crosslinked polymeric matrix of the microspheres is solely based on the base constituents a), b), c) and d) as defined above, in the abovementioned proportions of monomers and of transfer agent, no other base constituent being added to the reaction medium. It is thus obvious that the sum of the abovementioned proportions of monomers a), b) and c) must be equal to 100%.

Preferably, the hydrophilic monomer of formula (I) is chosen from the group consisting of N-vinylpyrrolidone, vinyl alcohol, 2-hydroxyethyl methacrylate, sec-butyl acrylate, n-butyl acrylate, t-butyl acrylate, t-butyl methacrylate, methyl methacrylate, N-dimethylaminoethyl (meth)acrylate, N, N-dimethylaminopropyl (meth)acrylate, t-butylaminoethyl (meth)acrylate, N, N-diethylaminoacrylate, poly(ethylene oxide) (meth)acrylate, methoxy poly(ethylene oxide) (meth)acrylate, butoxy poly(ethylene oxide) (meth)acrylate, poly(ethylene glycol) (meth)acrylate, methoxy poly(ethylene glycol) (meth)acrylate, butoxy poly(ethylene glycol) (meth)acrylate, poly(ethylene glycol) methyl ether methacrylate and their mixtures.

More advantageously, the hydrophilic monomer a) is poly(ethylene glycol) methyl ether methacrylate (m-PEGMA).

In the context of the present invention, the hydrophilic monomer a) is in particular added to the reaction mixture in an amount of from 20% to 90%, preferably from 30% to 80%, in a preferred way from 40% to 70%, in particular from 45% to 65%, per mole, with respect to the total number of moles of monomers. Thus, in the context of the present invention, the crosslinked matrix is in particular based on the hydrophilic monomer a) in an amount of from 20% to 90%, preferably from 30% to 80%, in a preferred way from 40% to 70%, in particular from 45% to 65%, per mole, with respect to the total number of moles of monomers.

Advantageously, the nonbiodegradable linear or branched hydrophilic crosslinking monomer exhibits (CH₂═(CR₁₆))CO— or (CH₂═(CR₁₆))CO—O— groups at its at least two ends, each R₁₆ independently representing H or a (C₁-C₆)alkyl.

In particular, the crosslinking agent is of following general formula (IIIa) or (IIIb):

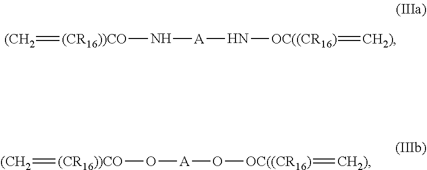

in which:
each R₁₆ independently represents H or a (C₁-C₆)alkyl; advantageously, the R₁₆ radicals are identical and represent H or (C₁-C₆)alkyl; and
A represents, alone or with at least one of the atoms to which it is bonded, a (C₁-C₆)alkylene, a polyethylene glycol (PEG), a polysiloxane, a poly(dimethylsiloxane) (PDMS), a polyglycerol ester (PGE) or a bisphenol A.

Advantageously, the crosslinking agent is of following general formula (IIa) or (IIb):

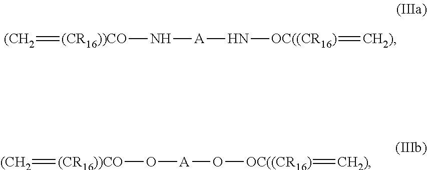

in which,
each R₁₆ independently represents H or a (C₁-C₆)alkyl; advantageously, the R₁₆ radicals are identical and represent H or (C₁-C₆)alkyl; and
A preferably represents, alone or with at least one of the atoms to which it is bonded, a (C₁-C₆)alkylene or a polyethylene glycol (PEG), preferably a polyethylene glycol (PEG).

In the context of the definitions of A above, the polyethylene glycol has a length varying from 200 to 10 000 g/mol, preferably from 200 to 2000 g/mol, more preferably from 500 to 1000 g/mol.

Mention may be made (without being limiting), as examples of crosslinking monomer which can be used in the context of the present invention, of: 1,4-butanediol diacrylate, pentaerythritol tetraacrylate, methylenebisacrylamide, glycerol 1,3-diglycerolate diacrylate and poly(ethylene glycol) dimethacrylate (PEGDMA).

Advantageously, the crosslinking monomer is poly(ethylene glycol) dimethacrylate (PEGDMA), the polyethylene glycol unit having a length varying from 200 to 10 000 g/mol, preferably from 200 to 2000 g/mol, more preferably from 500 to 1000 g/mol.

In the context of the present invention, the crosslinking monomer is in particular added to the reaction mixture in an amount of from 1% to 15%, preferably from 2% to 10%, in particular from 2% to 7%, more particularly from 2% to 5%, per mole, with respect to the total number of moles of monomers.

Advantageously, said chain transfer agent is chosen from the group consisting of monofunctional or polyfunctional thiols, and alkyl halides.

The alkyl halides which can be used as transfer agent include in particular bromotrichloromethane, tetrachloromethane and tetrabromomethane. Particularly advantageously, said chain transfer agent is an aliphatic or cycloaliphatic thiol typically having from 2 to approximately 24 carbon atoms, preferably from 2 to 12 carbon atoms, more preferentially 6 carbon atoms, and optionally having an additional functional group chosen from the amino, hydroxy and carboxy groups.

Advantageously, the transfer agent is chosen from thioglycolic acid, 2-mercaptoethanol, dodecanethiol, hexanethiol and their mixtures.

In the context of the present invention, the transfer agent is in particular added to the reaction mixture in an amount of from 0.1% to 10%, preferably from 0.5% to 8%, more advantageously from 1.5% to 6% and in particular from 1.5% to 4.5%, per mole, and in particular 3%, per mole, with respect to the number of moles of hydrophilic monomer a).

In another particular embodiment of the invention, the crosslinked polymeric matrix of the microspheres of the invention is in addition based:
on an ionized or ionizable monomer, and/or
on a colored monomer to render them visible to the naked eye, for example in order to confirm, before injection, that the suspension of microspheres is indeed homogeneous in the syringe and to control the rate of injection, and/or
on at least one agent visible in magnetic resonance imaging (MRI).

Said ionized or ionizable monomer, said colored monomer and said agent visible in MRI are in particular as defined in the application WO2021/069528, especially on pages 22-25.

In particular, the crosslinked polymeric matrix of the embolization microspheres of the invention can in addition be based on at least one ionized or ionizable monomer of the following formula (IV):

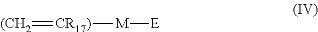

in which:
   R₁₇ represents H or a (C₁-C₆)alkyl;
   M represents a single bond or a divalent radical having from 1 to 20 carbon atoms;
   E represents a charged or ionizable group having 100 atoms at most, E advantageously being chosen from the group consisting of —COOH, —COO⁻, —SO₃H, —SO₃⁻, —PO₃H₂, —PO₃H⁻, —PO₃²⁻, —NR₁₈R₁₉ and —NR₂₀R₂₁R₂₂⁺,
   R₁₈, R₁₉, R₂₀, R₂₁ and R₂₂ represent, independently of one another, H or a (C₁-C₆)alkyl.

The term “ionized or ionizable group” is understood to mean, within the meaning of the present invention, a group which is charged or which can be found in the charged form (in the form of an ion), that is to say carrying at least one positive or negative charge, according to the pH of the medium. For example, the COOH group can be ionized in the COO-form and the NH₂ group can be found in the ionized form NH₃⁺.

The introduction of an ionized or ionizable monomer into the reaction mixture makes it possible to increase the hydrophilicity of the resultant microspheres, thus increasing the degree of swelling of said microspheres, further facilitating their injection via catheters and microcatheters. Moreover, the presence of an ionized or ionizable monomer makes possible the loading of active substances within the microsphere.

According to a preferred alternative form, the ionized or ionizable monomer is of following formula (IV-A):

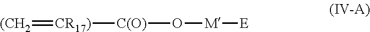

in which:
   R₁₇ and E are as defined above, and
   M′ is a hydrocarbon chain comprising from 1 to 20 carbon atoms.

Preferably, the ionized or ionizable monomer is a cationic monomer, advantageously chosen from the group consisting of methacrylic acid, (methacryloyloxy)ethylphosphorylcholine, 2-(dimethylamino)ethyl (meth)acrylate, 2-(diethylamino)ethyl (meth)acrylate, 11-methacryloyloxyundecylphosphonic acid and (2-((meth)acryloyloxy)ethyl)trimethylammonium chloride; advantageously, the cationic monomer is 2-(diethylamino)ethyl (meth)acrylate. Advantageously, the crosslinked matrix according to the invention is based on an abovementioned cationic monomer in amounts of between 1 mol % and 40 mol %, with respect to the total number of moles of monomers. Preferably, the crosslinked matrix according to the invention is based on ionized or ionizable monomer in amounts of between 5 mol % and 15 mol %, preferably 10 mol %, with respect to the total number of moles of monomers, when the resultant microspheres are not intended to be charged with an active substance. According to another embodiment, when the microspheres are intended to be charged with an active substance, the crosslinked matrix according to the invention is obtained by adding, to the reaction mixture, between 20 mol % and 40 mol %, preferably by adding, to the reaction mixture, from 20 mol % to 30 mol %, of ionized or ionizable monomer, with respect to the total number of moles of monomers.

In another advantageous embodiment, the ionized or ionizable monomer is an anionic monomer advantageously chosen from the group consisting of acrylic acid, methacrylic acid, 2-carboxyethyl acrylate, the 2-oligomers of carboxyethyl acrylate, 3-sulfopropyl (meth)acrylate, the potassium salt and the hydroxide of 2-((methacryloyloxy)ethyl)dimethyl (3-sulfopropyl) ammonium. Advantageously, the crosslinked matrix according to the invention is based on an abovementioned anionic monomer in amounts of between 1 mol % and 40 mol %, on the basis of the total amount of monomers. Preferably, the crosslinked matrix according to the invention is based on ionized or ionizable monomer in amounts of between 5 mol % and 15 mol %, preferably 10 mol %, on the basis of the total amount of monomers, when the resultant microspheres are not intended to be charged with an active substance. According to another embodiment, when the microspheres are intended to be charged with an active substance, the crosslinked matrix according to the invention is based on ionized or ionizable monomer in amounts of between 20 mol % and 40 mol %, preferably from 20 mol % to 30 mol %, of ionized or ionizable monomer, on the basis of the total amount of monomers.

Particularly advantageously, the ionized or ionizable monomer is methacrylic acid (MA). Advantageously, the crosslinked matrix according to the invention is based on methacrylic acid (MA) in amounts of between 10 mol % and 30 mol %, on the basis of the total amount of monomers.

Said crosslinked polymeric matrix can in addition be based on at least one colored monomer of following general formula (VI):

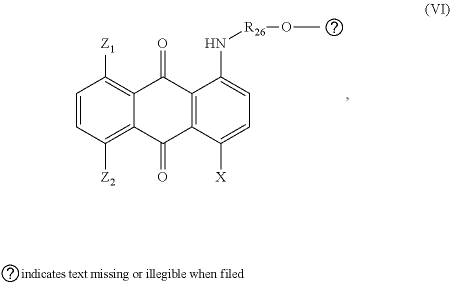

in which:
   Z₁ and Z₂ represent, independently of each other, H or OR₂₅, R₂₅ representing H or a (C₁-C₆)alkyl; advantageously, Z₁ and Z₂ represent H;
   X represents H or Cl, advantageously H;
   R₂₃ represents H or a (C₁-C₆)alkyl, advantageously a (C₁-C₆)alkyl, in particular a methyl; and
   R₂₄ represents a group chosen from linear or branched (C₁-C₆)alkylene, (C₅-C₃₆)arylene, (C₅-C₃₆)arylene-O—R₂₆, (C₅-C₃₆)heteroarylene and (C₅-C₃₆)heteroarylene-O—R₂₇, R₂₆ and R₂₇ representing a (C₁-C₆)alkyl or a (C₁-C₆)alkylene; advantageously, R₂₄ represents a —C₆H₄—O—(CH₂)₂—O or —C(CH₃)₂—CH₂—O group.

The term “(C_{X}-C_{Y})alkylene group” is understood to mean, within the meaning of the present invention, a linear or branched divalent hydrocarbon chain comprising from X to Y carbon atoms, X and Y being integers of between 1 and 36, preferably 1 and 18, in particular 1 and 6. Mention may be made, by way of examples, of the methylene, ethylene, propylene, butylene, pentylene or hexylene groups.

The term “(C_{X}-C_{Y})heteroarylene” is understood to mean, within the meaning of the present invention, a divalent aromatic group comprising from X to Y cyclic atoms including one or more heteroatoms, advantageously from 1 to 4 and more advantageously still 1 or 2, such as, for example, sulfur, nitrogen or oxygen atoms, the other cyclic atoms being carbon atoms. X and Y are integers of between 5 and 36, preferably 5 and 18, in particular 5 and 10.

The term “divalent radical” is understood to mean, within the meaning of the present invention, a radical having a valency of 2, that is to say having two covalent, polar covalent or ionic chemical bonds. Said radical can comprise, for example, carbon and/or oxygen atoms.

Advantageously, the colored monomer is of following formula (VIa) or (VIb):

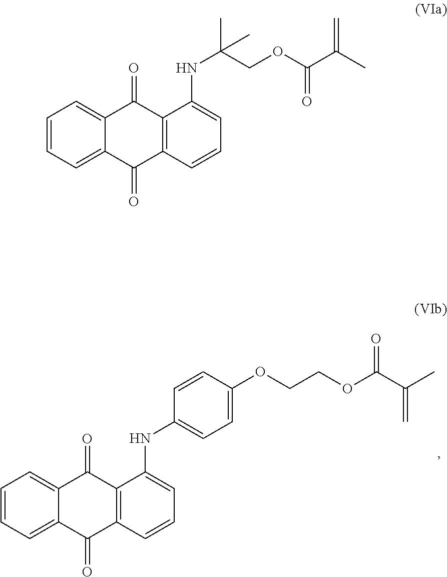 [SMILES: C=C(C)C(=O)OCC(C)(C)Nc1cccc2c1C(=O)c1ccccc1C2=O ; C=C(C)C(=O)OCCOc1ccc(Nc2cccc3c2C(=O)c2ccccc2C3=O)cc1]

More advantageously, the colored monomer is of formula (VIb) above.

In the context of the present invention, the colored monomer is in particular added to the reaction mixture in an amount of from 0% to 1%, preferably from 0% to 0.5%, more particularly from 0.02% to 0.2% and more particularly still from 0.04% to 0.1%, per mole, with respect to the total number of moles of monomers.

Said crosslinked polymeric matrix can additionally be based on elements visible in magnetic resonance imaging (MRI), such as iron oxide nanoparticles, gadolinium chelates or magnesium chelates, advantageously iron oxide nanoparticles.

In the context of the present invention, the elements visible in MRI are advantageously added to the reaction mixture in an amount of from 0% to 0.5%, preferably from 0.025% to 0.4%, more preferentially from 0.025% to 0.25%, in particular 0.05%, by weight of element visible in MRI per volume of organic phase, so as to obtain microspheres visible and quantifiable by MRI.

The crosslinked polymeric matrix of the embolization microspheres of the invention can be easily synthesized by numerous processes well known to a person skilled in the art. By way of example, it can be obtained by suspension polymerization as described in the application WO2021/069528, in particular on pages 27-29.

In a particular embodiment, the embolization microspheres according to the present invention are charged with active substances, thus making it possible to combine vascular occlusion and the delivery of an active principle. Said active substance can be chosen from a medicinal product, a diagnostic agent and macromolecules, as defined in the application WO2021/069528, in particular on pages 29-31.

Preferably, the microspheres according to the invention can be charged with an active substance chosen from anticancer agents, anti-inflammatory agents, local anesthetics, analgesics, antibiotics, steroids, antiseptics and their mixtures.

The anticancer agent is preferentially chosen from anthracyclines, such as doxorubicin, epirubicin or idarubicin, platinum complexes, compounds related to the anthracyclines, such as mitoxantrone and nemorubicin, antibiotics, such as mitomycin C (Ametycine®), bleomycin and actinomycin D, other antineoplastic compounds, such as irinotecan, 5-fluorouracil (Adrucil®), sorafenib (Nevaxar®), sunitinib (Sutent®), regorafenib, brivanib, orantinib, linsitinib, erlotinib, cabozantinib, foretinib, tivantinib, fotemustine, tauromustine (TCNU), carmustine, cytosine C, cyclophosphonamide, cytosine arabinoside (or cytarabine), paclitaxel, docetaxel, methotrexate, everolimus (Afinitor®), PEG-arginine deiminase, the tegafur/gimeracil/oteracil combination (Teysuno®), muparfostat, peretinoine, gemcitabine, bevacizumab (Avastin®), ramucirumab, floxuridine, immunostimulants, such as GM-CSF (granulocyte-macrophage colony-stimulating factor) and its recombinant forms: molgramostim or sargramostim (Leukine®), OK-432 (Picibanil®), interleukin-2, interleukin-4 and tumor necrosis factor-alpha (TNFalpha), antibodies, radioelements, complexes of these radioelements with chelates, nucleic acid sequences and a mixture of one or more of these compounds (preferentially a mixture of one or more anthracyclines).

Preferentially, the anticancer agent is chosen from anthracyclines, immunostimulants, platinum complexes, antineoplastics and their mixtures.

More preferentially still, the anticancer agent is chosen from anthracyclines, antibodies, antineoplastics and their mixtures.

The antibodies are, for example, chosen from the anti-PD-1 substances, anti-PD-L1 substances, anti-CTLA-4 substances, anti-CEA (CarcinoEmbryonic Antigen) substances or a mixture of these.

The anti-PD-1 substances are, for example, nivolumab or pembrolizumab.

The anti-PD-L1 substances are, for example, avelumab, durvalumab or atezolizumab.

The anti-CTLA-4 substances are, for example, ipilimumab or tremelimumab.

More advantageously still, the anticancer agent is chosen from the group consisting of paclitaxel, doxorubicin, epirubicin, idarubicin, irinotecan, GM-CSF (granulocyte-macrophage colony-stimulating factor), tumor necrosis factor-alpha (TNFalpha), antibodies and their mixtures.

Preferentially, the local anesthetic is chosen from lidocaine, bupivacaine and their mixtures.

The anti-inflammatory can be chosen from ibuprofen, niflumic acid, dexamethasone, naproxen and their mixtures.

In the context of the present invention, the microspheres can be charged, in particular by extemporaneous adsorption, with macromolecules chosen from the group consisting of enzymes, antibodies, cytokines, growth factors, clotting factors, hormones, plasmids, antisense oligonucleotides, siRNA, ribozymes, DNA enzyme (also called DNAzyme), aptamers, anti-inflammatory proteins, bone morphogenetic proteins (BMP), pro-angiogenic factors, vascular endothelial growth factors (VEGF) and TGF-beta, and angiogenesis inhibitors or antityrosine kinases and their mixtures.

The anti-inflammatory proteins are, for example, infliximab or rilonacept and their mixture.

The pro-angiogenic factors are, for example, fibroblast growth factors (FGF) and their mixture.

The angiogenesis inhibitors are, for example, bevacizumab, ramucirumab, nesvacumab, olaratumab, vanucizumab, rilotumumab, emibetuzumab, aflibercept, ficlatuzumab, pegaptanib and their mixtures.

The antityrosine kinases are, for example, lenvatinib, sorafenib, sunitinib, pazopanib, vandetanib, axitinib, regorafenib, cabozantinib, fruquintinib, nintedanib, anlotinib, motesanib, cediranib, sulfatinib, dovetinib, linifanib and their mixtures.

Advantageously, the microspheres can be charged with macromolecules chosen from antityrosine kinases, TGF-beta substances, angiogenesis inhibitors and their mixtures.

The active substance is typically adsorbed on the crosslinked matrix by noncovalent interactions, optionally in the presence of pharmaceutically acceptable excipient(s) well known to a person skilled in the art.

This particular way of trapping the active substances is called physical encapsulation. No particular requirement is imposed on the active substance to be charged.

Charging can be carried out by many processes well known to a person skilled in the art, such as passive adsorption (swelling of the crosslinked matrix in a solution of medicinal product) or by ionic interaction. These methods are, for example, described in the international application WO 2012/120138, in particular from page 22, line 20, to page 26, line 7. The effectiveness of the charging depends mainly on the compatibility between the two structures and/or favorable interactions.

Another subject matter of the invention relates to a pharmaceutical composition comprising embolization microspheres according to the invention, in combination with a pharmaceutically acceptable vehicle, advantageously for administration by injection.

An example of a pharmaceutically acceptable vehicle comprises, but without being limited thereto, water for injection, saline solution, also called physiological saline, starch, hydrogel, polyvinylpyrrolidone, polysaccharide, hyaluronic acid ester, plasma, a contrast agent for imaging by X-ray, by magnetic resonance or by ultrasonography, a buffering agent, a preservative, a gelling agent, glucose and/or a surface-active agent. Advantageously, the pharmaceutically acceptable vehicle is physiological saline, water for injection, a contrast agent for imaging by X-ray, by magnetic resonance or by ultrasonography, or their mixtures. More advantageously, the pharmaceutically acceptable vehicle is physiological saline, a contrast agent for imaging by X-ray, by magnetic resonance or by ultrasonography, or a mixture of physiological saline and of a contrast agent for imaging by X-ray, by magnetic resonance or by ultrasonography.

According to the present invention, the contrast agent is preferably a contrast agent for X-ray imaging. It advantageously concerns a water-soluble nonionic iodinated contrast agent, such as, for example, iobitridol (Xenetix®), iopamidol (Iopamiron®, Isovue®), iomeprol (Iomeron®), ioversol (Optiray®, Optiject®), iohexol (Omnipaque®), iopentol (Imagopaque®), ioxitol (Oxylan®), iopromide (Ultravist®), metrizamide (Amipaque®), iosarcol (Melitrast®), iotrolan (Isovist®), iodixanol (Visipaque®), iosimenol and iosimide (Univist®) and a mixture of these.

According to another embodiment, the contrast agent is a contrast agent for magnetic resonance imaging (MRI). It advantageously concerns gadolinium chelates (Dotarem®, Gadopiclenol).

According to another embodiment, the contrast agent is a contrast agent for imaging by ultrasonography. It advantageously concerns sulfur hexafluoride (Sonovue®).

In a particular embodiment of the present invention, the pharmaceutical composition comprises embolization microspheres according to the invention, in combination with physiological saline, said composition being intended to be mixed with at least one contrast agent for imaging by X-ray, magnetic by resonance by or ultrasonography as defined above, in particular for X-ray imaging, before administration by injection, such a mixture leading to the suspending of the microspheres according to the invention.

In a particular embodiment according to the invention, the pharmaceutical composition according to the invention comprises embolization microspheres according to the invention, in combination with a mixture of physiological saline and of a contrast agent as defined above, the physiological saline and the contrast agent being present in proportions of from 70/30 to 20/80, advantageously from 50/50 to 20/80, preferably 50/50.

The pharmaceutical composition must have a viscosity acceptable for injection.

The embolization microspheres according to the invention can, as was indicated above, be used for various biomedical purposes, which means that they must be compatible with the human body or with the body of a mammal. More particularly, suitable biomedical materials do not have hemolytic properties.

Another subject matter of the present invention is a kit comprising a pharmaceutical composition as defined above and at least one means of injection of said composition, for administration of said composition parenterally. According to the present invention, the term “means of injection” is understood to mean any means making possible administration parenterally. Advantageously, said means of injection is one or more syringes and/or one or more syringes which may be prefilled and/or one or more catheters or microcatheters for administration of said composition by injection.

Advantageously, the pharmaceutical composition present in said kit comprises the microspheres according to the present invention in combination with physiological saline, a contrast agent or their mixture. More advantageously, said pharmaceutical composition comprises the microspheres according to the present invention in combination with a mixture of physiological saline and of a contrast agent in proportions of between 80/20 and 0/100, advantageously of between 70/30 and 40/60, preferentially 50/50.

Advantageously, the means of injection present in the kit according to the invention is suitable for administration parenterally of the pharmaceutical composition according to the invention. Thus, the size of the syringe(s) or of the (micro) catheter(s) will be adapted as a function of the size of the microspheres according to the invention and of the volume to be injected for embolization. A person skilled in the art will know how to choose the appropriate means of injection. According to a preferred embodiment, said means of injection is the Vectorio® device as described in the applications WO2016/166346, WO2016/166339, WO2017/005914 and WO2017/081178.

Another subject matter of the present invention is a kit comprising, on the one hand, a pharmaceutical composition as defined above and, on the other hand, at least one contrast agent for imaging by X-ray, by magnetic resonance or by ultrasonography, and optionally at least one means of injection for administration parenterally. The means of injection is as defined above.

In said kit, the pharmaceutical composition and the contrast agent are packaged separately and are intended to be mixed just before administration by injection.

In said kit, the at least one contrast agent is as defined above in the description. In particular, the at least one contrast agent is a contrast agent for X-ray imaging as defined above in the description.

In said kit, the pharmaceutical composition advantageously comprises the microspheres according to the present invention in combination with a pharmaceutically acceptable vehicle for administration by injection. Said pharmaceutically acceptable vehicle can be, for example, but without being limited thereto, water for injection, physiological saline, starch, hydrogel, polyvinylpyrrolidone, polysaccharide, hyaluronic acid ester, glucose and/or plasma. Preferably, in said kit, the pharmaceutical composition advantageously comprises the microspheres according to the present invention in combination with physiological saline or water for injection.

In said kit, the pharmaceutical composition is advantageously packaged directly in a means of injection, in particular in a syringe, suitable for the injection of embolization microspheres parenterally.

In said kit, the contrast agent is advantageously packaged in a vial or directly in a means of injection, in particular a syringe, especially suitable for the injection of embolization microspheres parenterally.

In said kit, the pharmaceutically acceptable vehicle/contrast agent proportions are between 50/50 and 0/100, advantageously between 40/60 and 0/100, preferably from 30/70 to 0/100.

### EXAMPLES

### Example 1: Synthesis of MAETIP (Compound of Formula (A) According to the Invention)

### Equipment and Method

### Chemicals:

Magnesium sulfate (MgSO₄, anhydrous, 98%, Sigma-Aldrich, ref.: 230391, CAS: 10034-99-8)
2,4,6-Triiodophenol (95-98%, BLDpharm, ref.: A17145, CAS: 609-23-4)
2-[2-(2-Chloroethoxy) ethoxy]ethanol (95-98%, TCI, ref.: QF-8470, CAS: 5197-62-6)
Sodium iodide (NaI, >99%, Oakwood Chemical, ref.: QE-0904, CAS: 7681-82-5)
Sodium hydroxide (NaOH, anhydrous, >98%, Sigma-Aldrich, ref.: S8045, CAS: 1310-73-2)
Methacrylic anhydride (AM, >94%, Sigma-Aldrich, ref.: 276685, CAS: 760-93-0)
Triethylamine (TEA, >99.5%, Sigma-Aldrich, ref.: 471283, CAS: 121-44-8)

### Solvents:

Absolute ethanol (EtOH, 99.96%, VWR, ref.: 20821.310, CAS: 64-17-5)
Ethyl acetate (anhydrous, 99.8%, Sigma-Aldrich, ref.: 270989, CAS: 141-78-6)
Heptane (>99%, Sigma-Aldrich, ref.: 34873, CAS: 142-82-5)
Dichloromethane (DCM, anhydrous, >99.8%, Sigma-Aldrich, ref.: 270997, CAS: 75-09-2)
Distilled water (grade 2)

### Items of Equipment:

Magnetic hotplate stirrer (Heidolph, MR Hei-Standard)
Rotary evaporator (Buchi Rotavapor R-215)
Precision balance (d=0.1 mg, Sartorius)
Silica column: ChromatoFlash (Buchi)

### 1. O-alkylation

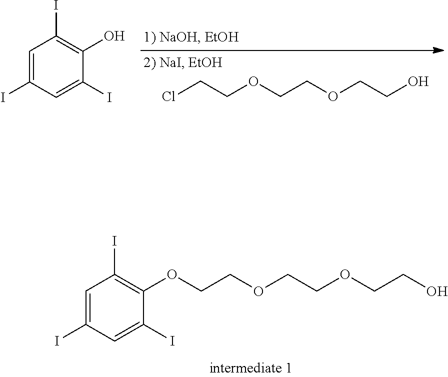 [SMILES: Oc1c(I)cc(I)cc1I ; OCCOCCOCCCl ; OCCOCCOCCOc1c(I)cc(I)cc1I]

Triiodophenol (200 mg, 0.42 mmol) is dissolved in 2.2 ml of ethanol in a 10 ml round-bottomed flask. NaOH (15 mg, 0.375 mmol) is added and then the mixture is stirred at ambient temperature for 30 minutes. Subsequent to the stirring, evaporation is carried out under vacuum until a slightly yellow solid is obtained.

NaI (57 mg, 0.375 mmol) and 2-[2-(2-chloroethoxy) ethoxy]ethanol (55 μl, 0.375 mmol) are added to a three-necked flask equipped with a reflux condenser and placed under nitrogen. The mixture is dissolved in 1.7 ml of ethanol until the NaI has completely dissolved. The triiodophenol derivative, dissolved beforehand in 0.7 ml of ethanol, is subsequently added. The reaction medium is heated at reflux for two and a half days, while ensuring a sufficiently high flow rate of water in the reflux condenser, so as to observe rapid condensation of the ethanol.

The progress of the reaction is monitored by TLC (thin layer chromatography): heptane/ethyl acetate 5/5.

At the end of the reaction, the reaction medium is evaporated under reduced pressure and then the solid obtained is dissolved in 6 ml of an NaOH (6M) solution. The aqueous phase is subsequently washed with DCM (dichloromethane) (3×7 ml). The organic phases are dried over MgSO₄. A yellowish solid is obtained. Purification is carried out on a silica column, via a heptane/ethyl acetate coelution system. Following the purification, 0.133 mg of a white solid is obtained.
Molar yield: 85%
UV Purity: 96%

### 2. Esterification of the Alcohol

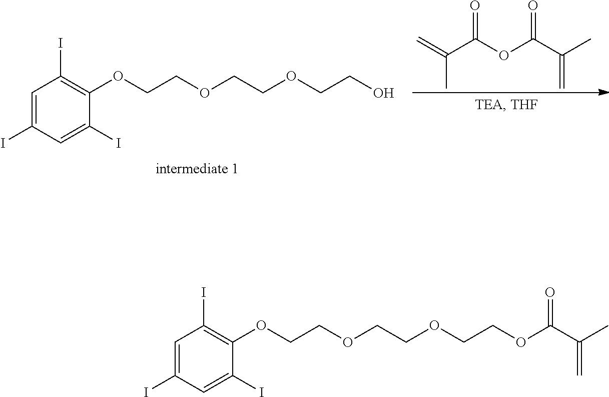 [SMILES: OCCOCCOCCOc1c(I)cc(I)cc1I ; C=C(C)C(=O)OC(=O)C(=C)C ; C=C(C)C(=O)OCCOCCOCCOc1c(I)cc(I)cc1I]

Intermediate 1 (0.133 mg, 0.22 mmol) is dissolved in 12 ml of anhydrous THF in a 25 ml three-necked flask equipped with a reflux condenser. TEA (triethylamine) (0.10 ml, 0.66 mmol) is added dropwise. The reaction medium is cooled to a temperature of less than 5° C. and then methacrylic anhydride (0.11 ml, 0.66 mmol) is added dropwise over a period of 5 minutes. Finally, the reaction medium is stirred at a temperature of less than 5° C. for one hour and then at reflux overnight.

The progress of the reaction is monitored by TLC: heptane/ethyl acetate 5/5.

At the end of the reaction, the reaction medium is brought back to ambient temperature and is then suspended in 90 ml of water for one hour. Washing is carried out with dichloromethane (3×20 ml). The organic phases are dried over MgSO₄ and then evaporated under reduced pressure until an orangey oil is obtained, which oil is subsequently purified on a silica column via a heptane/ethyl acetate coelution system. Following the purification, 89.7 mg of a transparent oil are obtained.
Molar yield: 74.5%
UV Purity: 96.6%

### 3. Conclusion

The synthesis of MAETIP was carried out with synthesis yields of approximately 54% (comparable with that of the synthesis of MAOETIB), with a purity of the final molecule of approximately 97%.

### Example 2: Synthesis by Oil-In-Water (Direct Phase) Suspension Polymerization of Microspheres According to the Invention Based on MAETIP, with a Size of 100-300 μm, 300-500 μm and 700-900 μm

The parameters of the syntheses are suited to the desired size of microspheres (see table 1).

### a) Preparation of the Aqueous Phase

An aqueous solution of hydrolyzed polyvinyl alcohol (PVA) and of sodium chloride is prepared according to the following protocol:
i) Dissolution of the PVA in 5 liters of apyrogenic water and stirring at 50° C. overnight.
ii) Addition of NaCl and stirring at ambient temperature for 4 hours.

### b) Preparation of the Organic Phase

v) Weighing of each reactant and of toluene.
w) Dissolution of AIBN in one volume of toluene.
x) Dissolution in one volume of toluene (in a different container) of poly(ethylene glycol) methyl ether methacrylate (m-PEG₃₀₀MA) (hydrophilic monomer), of poly(ethylene glycol) dimethacrylate (PEG₁₀₀₀DMA) (crosslinking agent), of methacrylic acid (MA) (ionizable monomer), of MAETIP (radiopaque monomer) and of the colorant, then addition of hexanethiol (transfer agent).
y) Addition of the AIBN solution obtained in stage w) to the solution of monomers of stage x).

### c) Synthesis of the Microspheres According to the Invention

The aqueous solution of PVA and of NaCl prepared in stage a) is poured into a reactor and heated to 50° C. The organic phase obtained in stage b) is subsequently introduced into the reactor. Stirring is applied with a stirrer of helical type in order to obtain droplets of dispersed phase of the desired diameter. The temperature is subsequently increased to 80° C. and stirring is maintained for 8 hours. The mixture is subsequently filtered with a 50 μm sieve and the microspheres are washed with acetone, then with ethanol and then with water before being sieved with sieves having a size of 50 μm, 100 μm, 300 μm and 500 μm, 700 μm, 900 μm and 1200 μm.

The main parameters of the synthesis are summarized in table 1 below according to the size of the microspheres.

TABLE 1

| Parameters | 100-300 μm | 300-500 μm | 700-900 μm |
|---|---|---|---|
| O/W (oil/water) ratio | 1/11 | 1/8 | 1/6 |
| Concentration by weight | 56% | 56% | 32% |
| of monomer in the organic | | | |
| phase | | | |
| Stirring rate (rpm) | 230 | 180 | 120 |
| Aqueous phase | 1% PVA | 0.25% | 0.25% |
| | (13-23 | PVA | PVA |
| | kDa)/3% | (30-70 | (30-70 |
| | NaCl | kDa)/7% | kDa)/7% |
| | | NaCl | NaCl |
| Total volume (ml) | 240 | 240 | 240 |
| Volume of the aqueous | 220 | 213 | 206 |
| phase (ml) | | | |
| n(PEG₃₀₀MA)/n(monomer | 64.98 | 64.98 | 54.98 |
| phase)* (%) | | | |
| n(PEG₁₀₀₀DMA/n(monomer | 5 | 5 | 5 |
| phase)* (%) | | | |
| n(MA)/n(monomer phase)* | 10 | 10 | 10 |
| n(MAETIP)/n(monomer | 20 | 20 | 30 |
| phase)* (%) | | | |
| n(hexanethiol)/n(PEG₃₀₀MA) | 3 | 3 | 3 |
| (%)* | | | |
| n(colorant)/n(monomer | 0.02 | 0.02 | 0.02 |
| phase)* (%) | | | |
| n(AIBN)/n(methacrylate | 1 | 1 | 1 |
| functions)* (%) | | | |

*n = number of moles

### Example 3: Measurement of the Suspension Times of the MS According to the Invention in Comparison with MS Comprising Another Radiopaque Monomer than MAETIP

The suspension times of the MS according to the invention synthesized in example 1 in an injection medium (50/50 physiological saline/iodinated contrast agent) were studied and compared with that of control MS comprising a radiopaque monomer other than MAETIP.

The control MS are synthesized according to the same protocol as that of example 1 with the same constituents, with the exception of the radiopaque monomer. The following radiopaque monomers are used in the control MS:
MAOETIB of formula:

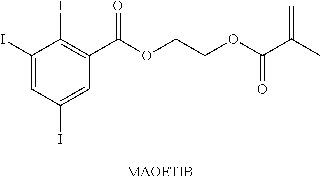 [SMILES: C=C(C)C(=O)OCCOC(=O)c1cc(I)cc(I)c1I]

The compound Vb described in the application WO 2021/069528 of formula:

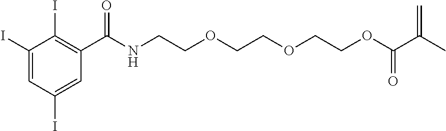 [SMILES: C=C(C)C(=O)OCCOCCOCCNC(=O)c1cc(I)cc(I)c1I]

The suspending was carried out using the “Falcon” method:

### Falcon Method:

| | | Suspension time | | | |
|---|---|---|---|---|---|
| Radiopaque | | (in seconds) | | | |
| monomer | Sample | n = 3 | | | Mean |
| MAOETIB | BF123 | 74 | 60 | 80 | 82 |
| (control) | BF124 | 88 | 94 | 99 | (±12) |
| | BF125 | 83 | 90 | 70 | |
| Compound Vb | BF126 | 115 | 100 | 125 | 174 |
| (control) | BF127 | 205 | 205 | 220 | (±47) |
| | BF128 | 210 | 210 | 180 | |
| MAETIP | BF129 | 320 | 315 | 330 | 297 |
| (invention) | BF130 | 323 | 318 | 305 | (±36) |
| | BF131 | 230 | 250 | 280 | |

1 ml of radiopaque microspheres is added to a Falcon® (or Axygen®) tube, the internal coating of which comprises 15 ml of polypropylene (of polystyrene), then the tube is made up to “water flower” (observation of an edge effect, so as to eject as much air as possible from the tube) with a 50/50 mixture of distilled water and of contrast agent;
the microspheres are suspended by successive inversions of the tube for 3 minutes;
the tube is placed at rest and the sedimentation (or creaming) time of the microspheres is measured.

### Results and Discussion

a) A first test of suspending the microspheres was carried out in Axygen® tubes according to the protocol described above. The results obtained are as follows:

### Table 2: Suspension Time of the MS According to the Falcon Method in Axygen® Tubes

TABLE 2
Suspension time of the MS according to the Falcon
method in Axygen ® tubes

| | | Suspension time | | | |
|---|---|---|---|---|---|
| Radiopaque | | (in seconds) | | | |
| monomer | Sample | n = 3 | | | Mean |
| MAOETIB | BF123 | 82 | 114 | 130 | 132 |
| (control) | BF124 | 108 | 150 | 160 | (±26) |
| | BF125 | 140 | 150 | 155 | |
| Compound Vb | BF126 | 150 | 195 | 200 | 251 |
| (control) | BF127 | 240 | 360 | 440 | (±94) |
| | BF128 | 170 | 240 | 265 | |
| MAETIP | BF129 | >600* | >600* | >600* | 571 |
| (invention) | BF130 | >600* | >600* | >600* | (±57) |
| | BF131 | 470 | 470 | >600* | |

It should be noted that, in each of the cases, sedimentation of the microspheres is observed at the end of the suspension in the injection medium.
b) A second test was carried out following the same parameters, in a Falcon® tube. The results obtained were as follows:

### Table 3: Suspension Time of the MS According to the Falcon Method in Falcon® Tubes

In each of the cases, sedimentation of the microspheres is observed at the end of the suspension in the injection medium.

For both tests, it is observed that the suspension time of the MS according to the invention is far greater than that of the control MS.

The results annotated by an “*” correspond to a suspension time of greater than 600 seconds. It was not considered relevant to measure the suspension time beyond 600 seconds, the results obtained by the suspension comprising the MS according to the invention being far greater than the results observed for the control microspheres.

1. A compound of following formula (A):

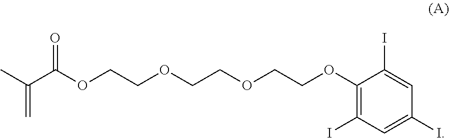 [SMILES: C=C(C)C(=O)OCCOCCOCCOc1c(I)cc(I)cc1I]

2. A method of medical imaging, comprising administering to a patient in need thereof an effective dose of the compound of formula (A) as defined in claim 1 as halogenated radiopaque monomer.

3. Radiopaque embolization microspheres comprising a crosslinked matrix, said matrix being based on at least:
a) from 20% to 90% of hydrophilic monomer chosen from N-vinylpyrrolidone and a monomer of following formula (I): 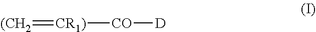
in which:
D represents O—Z or NH—Z, Z representing (C₁-C₆)alkyl, —(CR₂R₃)ₘ—CH₃, —(CH₂—CH₂—O)ₘ—H, —(CH₂—CH₂—O)ₘ—CH₃, —C(R₄OH)ₘ or —(CH₂)ₘ—NR₅R₆ with m representing an integer from 1 to 30;
R₁, R₂, R₃, R₄, R₅ and R₆ represent, independently of one another, H or a (C₁-C₆)alkyl;
b) from 5% to 50% of compound of following formula (A): 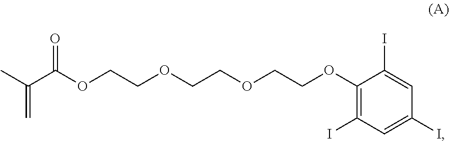 [SMILES: C=C(C)C(=O)OCCOCCOCCOc1c(I)cc(I)cc1I]
c) from 1% to 15% of nonbiodegradable linear or branched hydrophilic crosslinking monomer exhibiting (CH₂═(CR₁₆))— groups at each of its ends, each R₁₆ independently representing H or a (C₁-C₆)alkyl; and
d) from 0.1% to 10% of transfer agent being alkyl halides, cycloaliphatic thiols or aliphatic thiols, and optionally having another functional group chosen from amino, hydroxy and carboxy groups,
the percentages of the monomers a) to c) being given in moles with respect to the total number of moles of monomers, and the percentages of the compound d) being given in moles with respect to the number of moles of the hydrophilic monomer a).

4. The embolization microspheres of claim 3, wherein the matrix is based on the compound of general formula (A) in an amount of greater than 7% and of less than or equal to 50% per mole with respect to the total number of moles of monomers.

5. The embolization microspheres of claim 3, wherein the hydrophilic monomer a) is selected from the group consisting of N-vinylpyrrolidone, vinyl alcohol, 2-hydroxyethyl methacrylate, sec-butyl acrylate, n-butyl acrylate, t-butyl acrylate, t-butyl methacrylate, methyl methacrylate, N-dimethylaminoethyl (meth)acrylate, N,N-dimethylaminopropyl (meth)acrylate, t-butylaminoethyl (meth)acrylate, N,N-diethylaminoacrylate, poly(ethylene oxide) (meth)acrylate, methoxy poly(ethylene oxide) (meth)acrylate, butoxy poly(ethylene oxide) (meth)acrylate, poly(ethylene glycol) (meth)acrylate, methoxy poly(ethylene glycol) (meth)acrylate, butoxy poly(ethylene glycol) (meth)acrylate, poly(ethylene glycol) methyl ether methacrylate and mixtures thereof.

6. The embolization microspheres of claim 3, wherein the nonbiodegradable linear or branched hydrophilic crosslinking monomer c) exhibits (CH₂═(CR₁₆))CO— or (CH₂—(CR₁₆))CO—O— groups at its at least two ends, each R₁₆ independently representing H or a (C₁-C₆)alkyl.

7. The embolization microspheres of claim 3, wherein the transfer agent d) is thioglycolic acid, 2-mercaptoethanol, dodecanethiol, hexanethiol or mixtures thereof.

8. The embolization microspheres of claim 3, wherein the matrix is in addition based on at least one ionized or ionizable monomer of the following formula (IV): 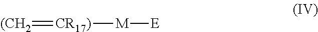
in which:
R₁₇ represents H or a (C₁-C₆)alkyl;
M represents a single bond or a divalent radical having from 1 to 20 carbon atoms;
E represents a charged or ionizable group having 100 atoms at most,
R₁₈, R₁₉, R₂₀, R₂₁ and R₂₂ represent, independently of one another, H or a (C₁-C₆)alkyl.

9. The embolization microspheres of claim 3, wherein the matrix is in addition based on at least one colored monomer of following general formula (VI): 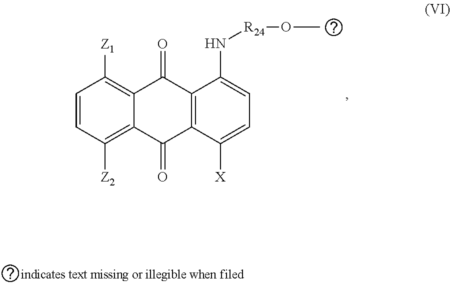
in which:
Z₁ and Z₂ represent, independently of each other, H or OR₂₅, R₂₅ representing H or a (C₁-C₆)alkyl;
X represents H or Cl;
R₂₃ represents H or a (C₁-C₆)alkyl, advantageously a (C₁-C₆)alkyl, in particular a methyl; and
R₂₄ represents a group chosen from linear or branched (C₁-C₆)alkylene, (C₅-C₃₆)arylene, (C₅-C₃₆)arylene-O—R₂₆, (C₅-C₃₆)heteroarylene and (C₅-C₃₆)heteroarylene-O—R₂₇, R₂₆ and R₂₇ representing a (C₁-C₆)alkyl or a (C₁-C₆)alkylene.

10. The embolization microspheres of claim 3, wherein the matrix is additionally based on elements visible in magnetic resonance imaging (MRI).

11. The microspheres of claim 8, charged with an active substance selected from the group consisting of anti-inflammatory agents, local anesthetics, analgesics, antibiotics, anticancer agents, steroids, antiseptics and mixture thereof.

12. The embolization microspheres of claim 8, charged with macromolecules selected from the group consisting of enzymes, antibodies, cytokines, growth factors, clotting factors, hormones, plasmids, antisense oligonucleotides, siRNA, ribozymes, DNA enzyme, aptamers, anti-inflammatory proteins, bone morphogenetic proteins (BMP), pro-angiogenic factors, vascular endothelial growth factors (VEGF) and TGF-beta, angiogenesis inhibitors, antityrosine kinases, and mixtures thereof.

13. A pharmaceutical composition comprising at least one embolization microsphere of claim 3, in combination with a pharmaceutically acceptable vehicle.

14. A kit comprising a pharmaceutical composition as defined in claim 13, in combination with a pharmaceutically acceptable vehicle, for parenteral administration, and at least one means of injection.

15. A kit comprising, on the one hand, a pharmaceutical composition as defined in claim 13 and, on the other hand, at least one contrast agent for imaging by X-ray, by magnetic resonance or by ultrasonography, and optionally at least one means of injection for administration parenterally, the pharmaceutical composition and the at least one contrast agent being packaged separately.

16. The embolization microspheres of claim 3, wherein the matrix is based on the compound of general formula (A) in an amount from 20% to 30% per mole with respect to the total number of moles of monomers.

17. The embolization microspheres of claim 3, wherein the hydrophilic monomer a) is poly(ethylene glycol) methyl ether methacrylate.

18. The embolization microspheres of claim 8, wherein in formula (IV), E is selected from the group consisting of —COOH, —COO⁻, —SO₃H, —SO₃⁻, —PO₃H₂, —PO₃H⁻, —PO₃²⁻, —NR₁₈R₁₉ and —NR₂₀R₂₁R₂₂⁺.

19. The embolization microspheres of claim 9, wherein in formula (VI), R₂₄ represents a —C₆H₄—O—(CH₂)₂—O or —C(CH₃)₂—CH₂—O group.

20. The embolization microspheres of claim 10, wherein the matrix is additionally based on iron oxide nanoparticles, gadolinium chelates or magnesium chelates.
